(19) United States
(12) Patent Application Publication
(10) Pub. No.: US 2008/0153906 A1
(43) Pub. Date: Jun. 26, 2008

(54) Water-Soluble Phenicol Prodrugs in a Lipophilic Vehicle System

(76) Inventors: Chander S. Celly, Colonia, NJ (US); Keith A. Freehauf, Stockton, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(21) Appl. No.: 11/953,978

(22) Filed: Dec. 11, 2007

Related U.S. Application Data
(60) Provisional application No. 60/874,798, filed on Dec. 13, 2006.
(60) Provisional application No. 60/874,860, filed on Dec. 13, 2006.
(60) Provisional application No. 60/874,864, filed on Dec. 13, 2006.

(51) Int. Cl. A61K 31/222

(52) U.S. Cl. 514/550

(57) ABSTRACT

The present invention discloses pharmaceutical compositions containing a suspension of a therapeutically effective amount of a water-soluble prodrug of a phenicol in a lipophilic vehicle system.

### CROSS-REFERENCE TO RELATED APPLICATIONS

This application is a non-provisional application that claims the benefit of U.S. provisional application No. 60/874,798 filed Dec. 13, 2006. U.S. provisional application No. 60/874,860 filed Dec. 13, 2006, and U.S. provisional application No. 60/874,864 filed Dec. 13, 2006, the contents of all of which are hereby incorporated by reference in their entireties.

### FIELD OF THE INVENTION

The present invention relates to compositions containing a phenicol prodrug in a lipophilic vehicle system. Corresponding pharmaceutical compositions and methods of administering these pharmaceutical compositions to animal subjects are also provided.

### BACKGROUND OF THE INVENTION

Florfenicol is a structural analog of thiamphenicol, which in turn is a derivative of chloramphenicol, [see, e.g., U.S. Pat. Nos. 4,235,892 and 5,352,832, the contents of which are hereby incorporated by reference in their entireties].

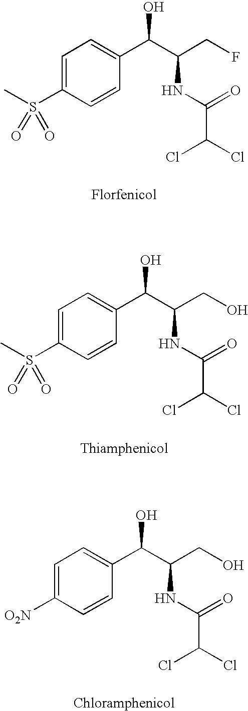

Such phenicols are broad spectrum antibiotics with activity against many gram-negative and gram-positive bacteria, including utility in the prevention and treatment of bacterial infections due to susceptible pathogens in birds, reptiles, fish, shellfish and mammals. One of florfenicol's primary uses is in the treatment of pneumonia and associated respiratory infections in cattle (often referred to generically as Bovine Respiratory Disease or BRD) caused by Mannheimia haemolytica, Pasteurella multocida and/or Haemophilus somnus, also known as Histophilus somni. It is also indicated in the treatment of, pododermatitis in cattle caused by Fusobacterium necrophorum and Bacterioides melaninogenicus; swine respiratory disease caused by Pasteurella multocida, Actinobacillus pleuropneumoniae, Streptococcus suis, Salmonella cholerasuis and/or Mycoplasma spp.; colibacillosis in chickens caused by Escherichia coli; enteric septicemia in catfish caused by Edwardsiella ictaluri; and furunculosis in salmon caused by Aeromonas salmonicida. Other genera of bacteria that have exhibited susceptibility to florfenicol include Enterobacter, Klebsiella, Staphylococcus, Enterococcus, Bordetella, Proteus and Shigella. In particular, chloramphenicol-resistant strains of organisms, such as K. pneumoniae, E. cloacae, S. typhus and E. coli, are susceptible to florfenicol.

Phenicols are most often administered to subjects either orally or parenterally, the latter being primarily subcutaneous, intramuscular or intravenous. Organic solvents are often employed to achieve the desired product concentration in a commercial formulation because of the limited water solubility of the phenicols.

Due to the need for economical, single-dose treatment in the veterinary setting, new phenicol prodrugs have been designed and synthesized that can be effectively delivered at high concentrations to the subject. Indeed, a considerable amount of work has been carried out on the production of water-soluble prodrugs of florfenicol, which lacks a primary alcohol group. For instance, esters of florfenicol with hydrocarbon carboxylic acids and with amino hydrocarbon carboxylic acids were described in U.S. Pat. No. 4,311,857 of Nagabhushan and U.S. Pat. No. 6,790,867 of Kohan et al. These patents describe primarily aliphatic esters of florfenicol, and also disclose esters produced from several amino acids, including the glycinate, ornithate and lysinate esters. Murthy et al. U.S. published patent application 2005/014828 A1, describes a number of esters of florfenicol with aliphatic carboxylic acids. Published U.S. patent application 2005/0182031 A1 (Hecker et al.) describes certain phosphate esters of florfenicol. All of the aforementioned patents and applications are hereby incorporated by reference herein, in their entireties.

Whereas the water-soluble prodrugs can be quite advantageous for administration in an aqueous media, for example, in drinking water, such aqueous solutions are not optimal for administration by injection. Indeed, one drawback that has been seen in some cases is that, while aqueous solutions of water-soluble phenicol prodrugs can provide a relatively high concentration of the phenicol parent compound immediately, or shortly after administration, the phenicol parent compound is quickly depleted from the circulation. In general, success of the prodrug approach requires assurance that the prodrug does not release the parent drug prematurely, either in the dosing solution or in the subject. Therefore, there remains a need for a form of phenicol, especially florfenicol, that is capable of maintaining effective plasma antibiotic levels for prolonged periods of time, in order to achieve improved economies in administration, e.g., to more readily provide single dose treatment, particularly in a veterinary setting.

It should be noted that the citation of any reference herein should not be construed that such reference is available as part of the “prior art” to the instant application.)

### BRIEF SUMMARY OF THE INVENTION

This invention provides liquid formulations or compositions containing water-soluble prodrugs of phenicols that are suitable for, and advantageous in, injection into subjects, including both subcutaneous and intramuscular injection.

More specifically this invention provides compositions containing a therapeutically effective amount of a water-soluble prodrug of a phenicol, wherein the compositions comprise a suspension of the phenicol prodrug in a lipophilic vehicle.

The invention further comprises a method of administering a phenicol to a subject comprising injecting said subject with a composition comprising a suspension of a therapeutically effective amount of a prodrug of said phenicol in a lipophilic vehicle. The pharmaceutical compositions of the present invention can be administered to animals or fish in prophylactically-effective amounts and/or for metaphylaxis, as a need and/or the practice merits. Corresponding methods of administering prophylactically-effective amounts of the pharmaceutical compositions of the present invention and/or for metaphylaxis, as a need and/or the practice merits, are also provided by the present invention. The present invention also provides methods of treating or preventing a disease or disorder in an animal in need thereof.

Additional features of this invention will be apparent from the more detailed description that follows.

### DETAILED DESCRIPTION OF THE INVENTION

This invention relates to compositions or formulations that comprise a suspension of a prodrug of a phenicol in a lipophilic vehicle, and methods of using them by administration, for example, injection, to a subject, as described below. In a particular aspect of the present invention, the subject is a non-human subject.

The phenicols (sometimes referred to herein as the “parent compound”) are a class of compounds that have the general chemical formula:

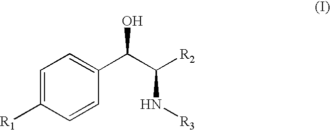

in which:
R₁ is selected from the group consisting of

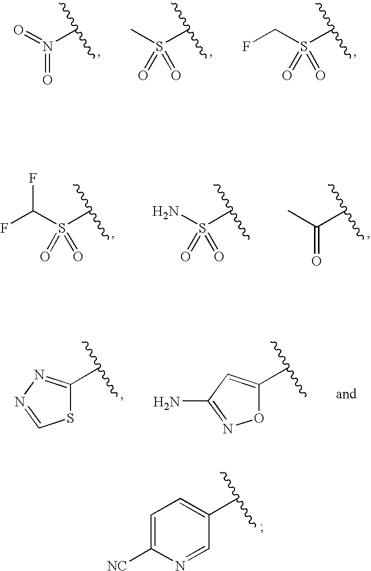

R₂ is selected from the group consisting of hydroxymethyl, fluoromethyl, difluoromethyl, and trifluoromethyl;
R₃ is selected from the group consisting of dichloromethyl, difluoromethyl chlorofluoromethyl, chloromethyl, methyl, cyanomethyl, azidomethyl, and aminomethyl;
and the pharmaceutically acceptable salts thereof.

When R₁ is nitro, R₃ is dichloromethyl and R₂ is hydroxymethyl, the phenicol is chloramphenicol. When R₁ is methylsulfonyl, R₃ is dichloromethyl, and R₂ is hydroxymethyl, the phenicol is thiamphenicol. When R₁ is methylsulfonyl, R₃ is dichloromethyl, and R₂ is fluoromethyl, the phenicol is florfenicol. When R₁, R₃ and/or R₂ are other possibilities listed for these substituents, the phenicol is an analog of one or another of these known phenicols.

Pharmaceutically acceptable salts of the phenicols (including analogs) described above include hydrochloride, hydrobromide, methanesulfonate, sulfate, 2-hydroxyethylsulfate, citrate, and phosphate.

Prodrugs of the phenicols that may be included in the compositions or formulations of this invention include those known water-soluble prodrugs of phenicols [all of the following patents and patent applications are hereby incorporated herein in their entirety], such as the esters of carboxylic acids and amino acids described in U.S. Pat. Nos. 3,740,411 and 3,770,889 (both of Akiyama et al.), 3,405,165 and 3,475,470 (both of Rebstock et al.), 4,311,857 of Nagabhushan and 6,790,867 of Kohan et al.; U.S. published patent application 2005/014828 of Murthy et al.; phosphate esters of florfenicol described in U.S. Pat. No. 7,153,842 of Hecker et al.; and carbonate esters of florfenicol described in U.S. patent publication 2007/0155799 filed Dec. 18, 2006.

For instance, the prodrug may be a compound having the formula

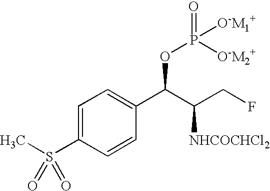

wherein M₁⁺ and M₂⁺ are H⁺ or a pharmaceutically-acceptable mono-cation, or taken together, are a pharmaceutically-acceptable di-cation, as disclosed in U.S. Pat. No. 7,153,842, the contents of which is hereby incorporated by reference in its entirety.

In certain embodiments of the compound of Formula (I), M₁⁺ and M₂⁺ are independently selected from the group consisting of H⁺, Na⁺, NH₄⁺ and K⁺. In one particular embodiment, one of M₁⁺ or M₂⁺ is H⁺, whereas the other is Na⁺. In another particular embodiment, M₁⁺ and M₂⁺ are both Na⁺.

In addition, the invention may be used to prepare compositions of water-soluble prodrugs of phenicols that are discovered at a later time.

In preferred embodiments the phenicol prodrugs comprise certain new nitrogen-containing esters of the phenicols and their analogs, particularly charged nitrogen-containing esters. These are described below, and also in U.S. provisional patent applications 60/874,860 and 60/874,864.

One series of these new phenicol prodrugs comprises prodrugs for florfenicol and analogs thereof, and has the general formula

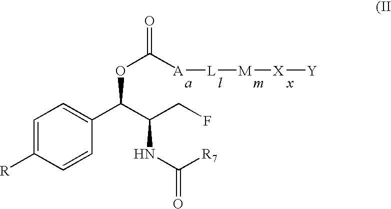

in which;
R is selected from the group consisting of

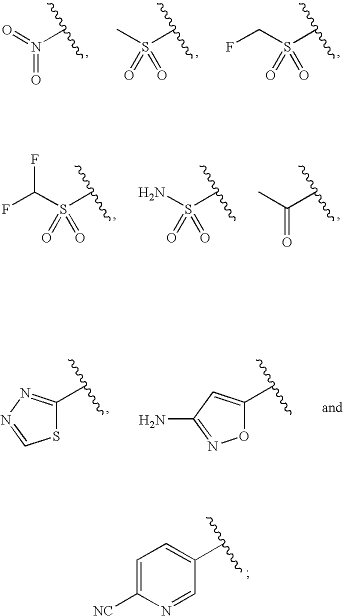

A is oxygen and a is zero or 1;
L is (a) CH₂ and l is an integer from 1 to 6; (b) CHR₁ where R₁ is an amino acid side chain and l is 1; or (c) CHR₁NHC(O)CH(NH₂)R₂ where R₁ and R₂ are amino acid side chains and l is 1;
M is (a) oxygen or sulfur and m is zero or one; (b) CH₂ and m is zero or an integer from 1 to 4; or (c) NH and m is 1;
X is (a) CH₂ and x is zero or an integer from 1 to 4; or (b) C(O) and x is 1; and Y is (a) NH₂; (b) NHRₓ, where Rₓ is methyl, ethyl, n-propyl or isopropyl; (c) NR_{y}R_{z}, where R_{y}, and R_{z}, are independently hydrogen, methyl, ethyl, n-propyl or isopropyl, or R_{y} and R_{z} taken together form a C₂-C₅ alkylene chain, or a C₂-C₄ alkylene chain further including a nitrogen or oxygen heteroatom in said chain; (d) C(═NH)NH₂; (e) N⁺R₄R₅R₆ where R₄, R₅ and R₆ are independently hydrogen, methyl or ethyl, or R₄ and R₅ taken together form a C₂-C₅ alkylene chain, or a C₂-C₄ alkylene chain further including a nitrogen or oxygen heteroatom in said chain; (f) pyridinium; (g) N-methyl or N-ethyl pyridinium; (h) N′-3-methyl-N-1-imidazolium; (i) a phenyl group substituted by a group having the formula NR₄R₅ or N⁺R₄R₅R₆ where R₄, R₅ and R₆ are as defined above; or (j) NH—CR₃(═NH) where R₃ is hydrogen, methyl or amino;
and R₇ is selected from the group consisting of dichloromethyl, difluoromethyl, trifluoromethyl, cyanomethyl, azidomethyl, and aminomethyl;
provided that the group AₐLₗMₘXₓY is other than an alpha-N-unfunctionalized glycine, ornithine or lysine residue;
and pharmaceutically acceptable salts thereof.

In a particular embodiment of these compounds, the nitrogen atom of a prodrug moiety is a charged atom. Furthermore in order to modulate the rate of hydrolysis of ester pro-moieties containing such a charged nitrogen atom or a sufficiently basic nitrogen atom assuring that the prodrug exists predominantly in charged form at the physiological pH, the nitrogen atom can be placed at a distance away from the carbonyl bond of the ester. The same effect can be achieved in carbonate derivatives containing a charged nitrogen atom by attaching the charged nitrogen atom further away from the hydrolyzable carbonate functionality. A charged nitrogen atom distance of at least two atoms, or at least three atoms, away from the carbonyl carbon atom of the ester or carbonate group to be hydrolyzed in the release of the parent drug is satisfactory for achieving the desired hydrolytic stability of the ester or carbonate.

Preferred compounds of this invention have the formula (III:

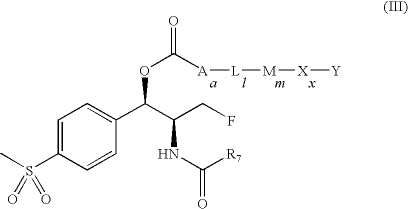

in which:
A is oxygen and a is zero or 1;
L is (a) CH₂ and l is an integer from 1 to 5 or (b) CHR₁, where R₁ is an amino acid side chain and l is 1; or (c) CHR₁NHC(O)CH(NH₂)R₂ where R₂ is an amino acid side chain and l is 1;
M is (a) oxygen and m is zero or one; (b) CH₂ and m is zero or an integer from 1 to 4; or (c) NH and m is 1;
X is (a) CH₂ and x is zero or an integer from 1 to 4; or (b) C(O) and x is 1; and
Y is (a) NH₂; (b) NHRₓ where Rₓ is methyl, ethyl, n-propyl or isopropyl; (c) NR_{y}R_{z} where R_{y} and R_{z} are independently hydrogen, methyl, ethyl, n-propyl or isopropyl; (d) C(═NH)NH₂; (e) N⁺R₄R₅R₆ where R₄, R₅ and R₆ are independently hydrogen, methyl or ethyl; N-pyridinium; (g) N′-methyl-N-1-imidazolium; or (h) NH—CR₃(═NH) where R₃ is hydrogen, methyl or amino;
and R₇ is selected from the group consisting of dichloromethyl, difluoromethyl, trifluoromethyl, cyanomethyl, azidomethyl, and aminomethyl; (with R₇ preferably being dichloromethyl)
provided that the sum of a+l+m+x is from 2 to 6 and preferably from 3 to 6;
provided that if a is 1, then M is (CH₂)ₘ;
and provided that the group AₐLₗMₘXₓY is other than an alpha-N-unfunctionalized glycine, ornithine or lysine residue;
and pharmaceutically acceptable salts thereof.

Particular compounds are those of Formula (II) or (III) in which Y includes a positively charged nitrogen atom, i.e. Y is a group N⁺R₄R₅R₆ where R₄, R₅ and R₆ are independently hydrogen, methyl or ethyl such as NH₃⁺, N⁺H₂(CH₃), N⁺H(CH₃)₂, N⁺(CH₃)₃, N⁺H₂(C₂H₅), N⁺H(C₂H₅)₂, N⁺(C₂H₅)₃, I—NH⁺-3-methylimidazolium. Compounds in which a is zero are esters; those in which a is 1 are carbonates. Other particular compounds are those of formula (I) or (I)) in which the group AₐLₗMₘXₓY is other than an alpha-N-unfunctionalized residue of an alpha-amino acid or of an aromatic alpha-amino acid (for example, the group AₐLₗMₘXₓY is not an alpha-N-unfunctionalized phenylalanine residue), or the group AₐLₗMₘXₓY is other than an alpha-N-unfunctionalized alpha-amino acid, or the group AₐLₗMₘXₓY is other than an alpha-amino acid residue.

Compounds of Formulas (II and (III) include compounds in which the group
Aₐ-Lₗ-Mₘ-Xₓ—Y_{y} is, for instance as shown immediately below):
   1. Dipeptide esters R=H, amino acid sidechain

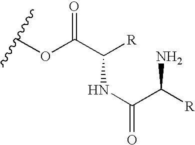

2. Omega amino esters (n=2-6) R=H, Me, Et

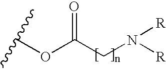

3. Omega amidino esters N-linked (n=2-6) R=H, Me

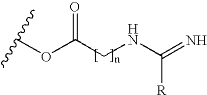

4. Omega amidino esters, C-linked (n=2-6)

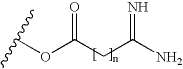

5. Omega guanidine esters (n=2-6)

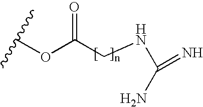

6. Omega amino acid esters-cyclic amines (n₁=1-6; n₂=2-4)

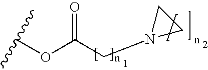

7. Omega quaternary ammonium esters (n=1-6) R=Me, Et

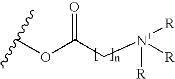

8. Omega quaternary ammonium esters-cyclic amines (n₁=2-6; n₂=2-4; R=Me, Et)

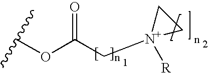

9. Omega quaternary ammonium esters-cyclic diamines (n=2-6; R=H, Me, Et)

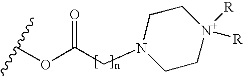

10. Omega pyridinium salts, C-linked; (n=2-6; R=Me, Et)

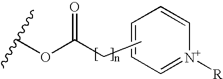

11. Omega imidazolium salts (n=2-6; R=Me, Et)

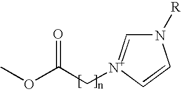

12. Aminomethyl benzoates (R=Me, Et)

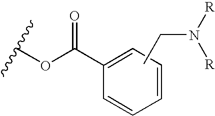

13. Quaternary salts of aminomethyl benzoates (R=Me, Et)

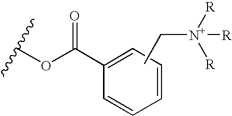

For types 2-11, also

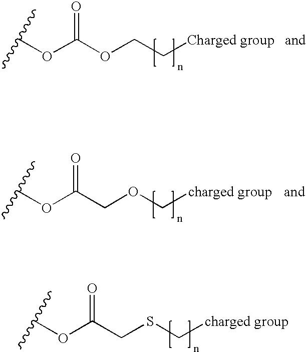

versions, where “charged group” represents a positively charged nitrogen-containing moiety of the type indicated.

Compounds of Formula (III) include, in general, and as exemplified below;
A. Carbonates with a terminal amine functionality
B. Esters with an additional alpha-heteroatom (O,S) in the linker; for instance —C(O)CH₂OCH₂CH₂NH₂, C(O)CH₂SCH₂CH₂NH₂
C. Esters with an additional alpha-heteroatom (N)—which are amino acid derivatives but do not bear a protonable amine at the alpha position; these are dipeptides—for example—
—C(O)CH₂NHC(O)CH₂NH₂ and —C(O)CH(Me)NHC(O)CH(Me)NH₂
D. Esters with quaternary nitrogen atoms removed at least two methylene groups away from the carbonyl group, for example C(O)CH₂CH₂CH₂—N-methylimidazolium and C(O)CH₂CH₂CH₂N⁺Me₃.

Some preferred compounds of formulas (II) and (III) are shown below:

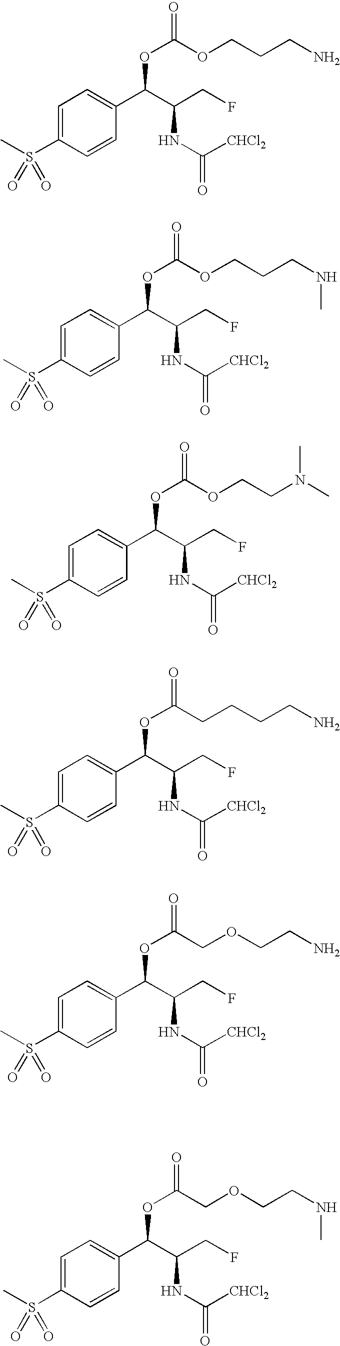

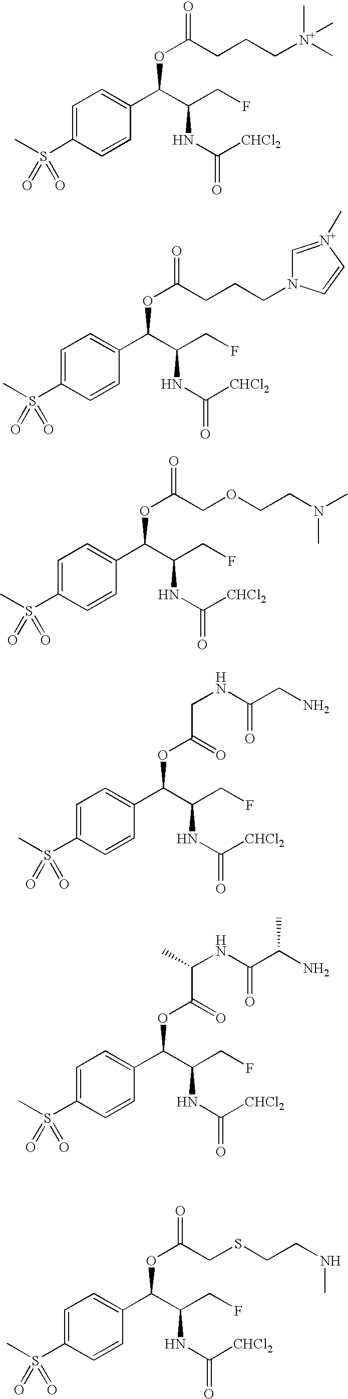

In one process for making the above, compounds of Formulas (II) and (III) that are esters may be prepared by reacting florfenicol or a florfenicol analog with a carboxylic acid or a derivative thereof having a terminal group W which represents a protected primary or secondary amine that is later deprotected to a free amine, a tertiary amine, or a group that is later manipulated into a required charged nitrogen functionality. A commercially available activated derivative of the carboxylic acid may be used for the formation of the ester; it may be prepared in a separate reaction step or it may be prepared in situ in presence of the florfenicol or florfenicol analog

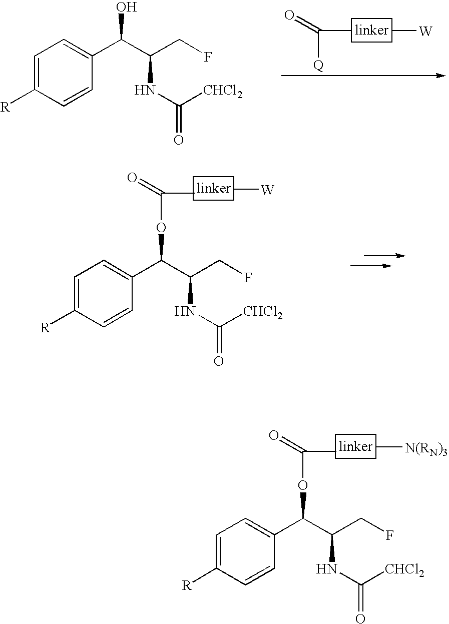

Compounds that are carbonates are prepared by reacting the florfenicol or florfenicol analog with a derivative of an alkoxycarbonic acid, for example a chloroformate having a terminal group W which represents a protected primary or secondary amine that is later deprotected to a free amine, a tertiary amine, or a group that is later manipulated into a required charged nitrogen functionality:

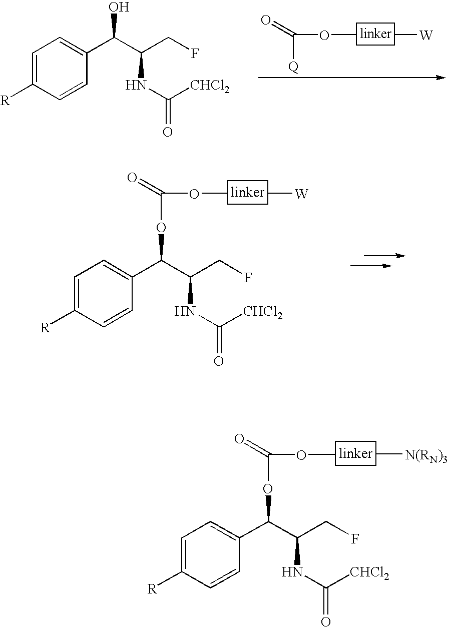

The necessary acids or chloroformates, if not commercially available, can readily be prepared by methods known to those in the art. Appropriate reaction conditions, solvents, etc., are exemplified below.

As shown in the above reaction schemes the secondary alcohol functionality of florfenicol or a florfenicol analog is reacted with the activated carboxylic acid or alkoxy carbonic acid reagents by nucleophilic displacement of group Q. Most common reagents of this type utilize Q=chlorine but many other leaving groups known in the art may be also employed.

As alternative to chloroformates (Q=chlorine), other reagents with different leaving groups Q may be used for preparation of carbonates of florfenicol alcohols. Representative references are cited for each leaving group, each incorporated by reference herein.

The reaction may be facilitated by the addition of a catalyst like a trialkylamine, pyridine, a 4-alkylpyridine, a 4-diaminoalkyl pyridine or a combination thereof. Formation of the initial ester or carbonate intermediate can be conveniently performed in a variety of solvents. Suitable solvents include, for example, chlorinated solvents such as dichloromethane and 1,2-dichloroethane; ester solvents such as ethyl acetate, isopropyl acetate, isoamyl acetate, ethylene glycol diacetate, propylene glycol diacetate, glycerol triacetate; monoether solvents such as diethyl ether, disopropyl ether, methyl tert-butyl ether; polyether solvents such as ethylene glycol ethers, dimethyl ethylene glycol ether, diethylene glycol ethers: diethylene glycol dimethyl ether, diethylene glycol diethyl ether; formaldehyde acetal ethers such as dimethoxymethane, diethoxymethane, dibutoxyethane; cyclic ethers such as tetrahydrofuran, 1,3-dioxolane, 1,4-dioxane; ketone solvents such as acetone, methyl ethyl ketone, methyl isobutyl ketone; mixed ether/ester solvents as represented by monoethers of ethylene and diethylene glycol such as 2-methoxyethyl acetate, 2-ethoxyethyl acetate, 2-(methoxy-ethoxy)ethyl acetate, and 2-(ethoxy-ethoxy)ethyl acetate.

Another type of prodrug comprises novel prodrugs of chloramphenicol or thiamphenicol or an analog of either, having the formula (IV):

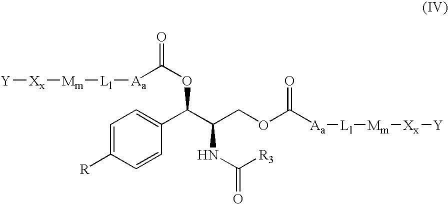

in which:
R is selected from the group consisting of

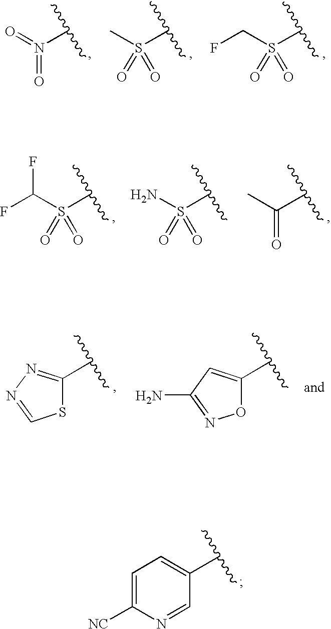

A is oxygen and a is zero or 1;
L is (a) CH₂ and l is an integer from 1 to 6; (b) CHR₁ where R₁ is an amino acid side chain and l is 1; or (c) CHR₁NHC(O)CH(NH₂)R₂ where R₁ and R₂ are amino acid side chains and l is 1;
M is (a) oxygen or sulfur and m is zero or one; (b) CH₂ and m is zero or an integer from 1 to 4; or (c) NH and m is 1;
X is (a) CH₂ and x is zero or an integer from 1 to 4; or (b) C(O) and x is 1; and
Y is (a) NH₂, (b) NHRₓ where Rₓ is methyl, ethyl, n-propyl or isopropyl; (c) NR_{y}R_{z} where R_{y} and R_{z} are independently hydrogen, methyl, ethyl, n-propyl or isopropyl, or R_{y} and R_{z} taken together form a C₂-C₅ alkylene chain, or a C₂-C₄ alkylene chain further including a nitrogen or oxygen heteroatom in said chain; (d) C(═NH)NH₂; (e) N⁺R₄R₅R₆ where R₄, R₅ and R₆ are independently hydrogen, methyl or ethyl or R₄ and R₅ taken together form a C₂-C₅ alkylene chain, or a C₂-C₄ alkylene chain further including a nitrogen or oxygen heteroatom in said chain; (f) pyridinium; (g) N-methyl or N-ethyl pyridinium; (h) N′-3-methyl-N-1-imidazolium; (i) a phenyl group substituted by a group having the formula NR₄R₅ or N⁺R₄R₅R₆ where R₄, R₅ and R₆ are as defined above; or (j) NH—CR₈(═NH) where R₈ is hydrogen, methyl or amino; and
R₃ is selected from the group consisting of dichloromethyl, difluoromethyl, chlorofluoromethyl, chloromethyl, methyl, cyanomethyl, azidomethyl, and aminomethyl;
and pharmaceutically acceptable salts thereof.

In a particular embodiment of these compounds, the nitrogen atom of a prodrug moiety is a charged atom. Furthermore, in order to modulate the rate of hydrolysis of ester promoieties containing such a charged nitrogen atom or a sufficiently basic nitrogen atom assuring that the prodrug exists predominantly in charged form at the physiological pH, the nitrogen atom can be placed at a distance away from the carbonyl bond of the ester. The same effect can be achieved in carbonate derivatives containing a charged nitrogen atom by attaching the charged nitrogen atom further away from the hydrolyzable carbonate functionality. A charged nitrogen atom distance of at least two atoms, or at least three atoms, away from the carbonyl carbon atom of the ester or carbonate group to be hydrolyzed in the release of the parent drug is satisfactory for achieving the desired hydrolytic stability of the ester or carbonate.

Some preferred compounds of Formula (IV) include those in which R is nitro or methylsulfonyl and R₃ is hydroxymethyl, and their pharmaceutically acceptable salts. In some preferred compounds the two groups Aₐ-Lₗ-Mₘ-Xₓ—Y are identical; in others they may be different.

Other preferred compounds are those of Formula (IV) in which Y includes a positively charged nitrogen atom, i.e. Y is a group N⁺R₄R₅R₆ where R₄, R₅ and R₆ are independently hydrogen, methyl or ethyl such as NH₃⁺, N⁺H₂CH₃ or N⁺(CH₃)₃. Compounds in which a is zero are esters, those in which a is 1 are carbonates.

Compounds of Formula (IV) include compounds in which the group
Aₐ-Lₗ-Mₘ-Xₓ—Y is, for instance as shown below:
   1. Dipeptide esters R=H, amino acid sidechain

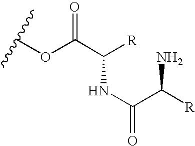

2. Omega amino esters (n=2-6) R=H, Me, Et

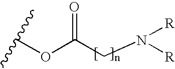

3. Omega amidino esters N-linked (n=2-6) R=H, Me

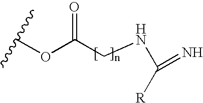

4. Omega amidino esters, C-linked (n=2-6)

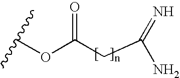

5. Omega guanidine esters (n=2-6)

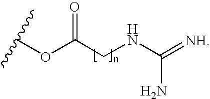

6. Omega amino acid esters-cyclic amines (n₁=1-6; n₂=2-4)

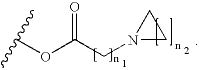

7. Omega quaternary ammonium esters (n=1-6) R=Me, Et

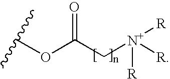

8. Omega quaternary ammonium esters-cyclic amines (n₁=1-6; n₂=2-4; R=Me, Et)

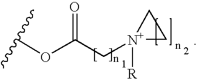

9. Omega quaternary ammonium esters-cyclic diamines (n=2-6: R=H, Me, Et)

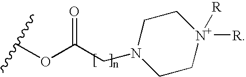

10. Omega pyridinium salts, C-linked; (n=2-6; R=Me, Et)

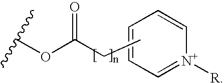

11. Omega imidazolium salts (n=2-6; R=Me, Et)

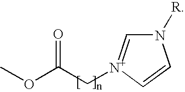

12. Aminomethyl benzoates R=Me, Et

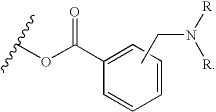

13. Quaternary salts of aminomethyl benzoates R=Me, Et

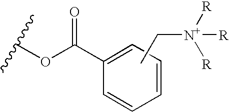

For types 2-11, also

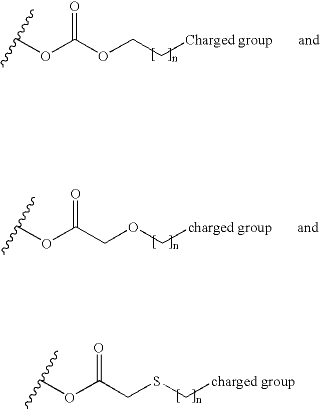

versions, where “charged group” represents a positively charged nitrogen-containing moiety of the type indicated.

Compounds of Formula (IV) include, in general, and as exemplified below:
A. Carbonates with a terminal amine functionality
B. Esters with an additional alpha-heteroatom (O,S) in the linker; for instance —COCH₂OCH₂CH₂NH₂ or C(O)CH₂SCH₂CH₂NH₂
C. Esters with an additional alpha-heteroatom (N)—which are amino acid derivatives but do not bear a protonable amine at the alpha position; these are dipeptides—for example—
   —C(O)CH₂NHCOCH₂NH₂ and —C(O)CH(Me)NHCOCH(Me)NH₂
D. Esters with quaternary nitrogen atoms removed at least two methylene groups away from the carbonyl group, for example C(O)CH₂CH₂CH₂—N-methylimidazolium and C(O)CH₂CH₂CH₂N⁺Me₃.

In one process, compounds of Formula (IV) that are esters may be prepared by reacting chloramphenicol or thiamphenicol or an analog of either with a carboxylic acid or a derivative thereof having a terminal group W which represents a protected primary or secondary amine that is later deprotected to a free amine, a tertiary amine, or a group that is later manipulated into a required charged nitrogen functionality. A commercially available activated derivative of the carboxylic acid may be used for the formation of the ester; it may be prepared in a separate reaction step or it may be prepared in situ in presence of chloramphenicol or thiamphenicol, or analog

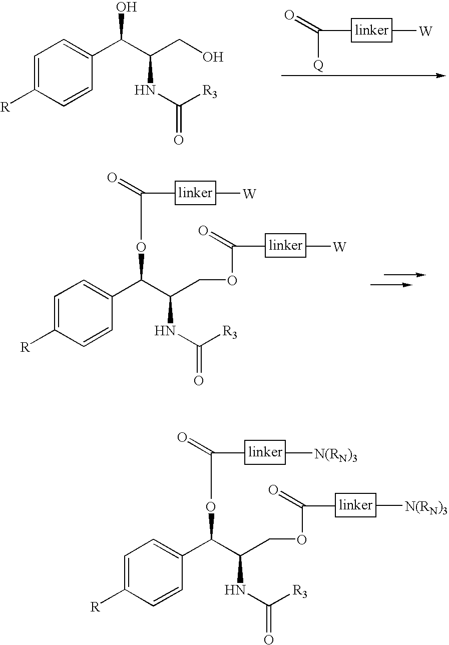

Compounds of Formula (IV) that are carbonates are prepared by reacting the chloramphenicol or thiamphenicol, or analog thereof, with a derivative of an alkoxycarbonic acid, or example a chloroformate having a terminal group W which represents a protected primary or secondary amine that is later deprotected to a free amine, a tertiary amine, or a group that is later manipulated into a required charged nitrogen functionality:

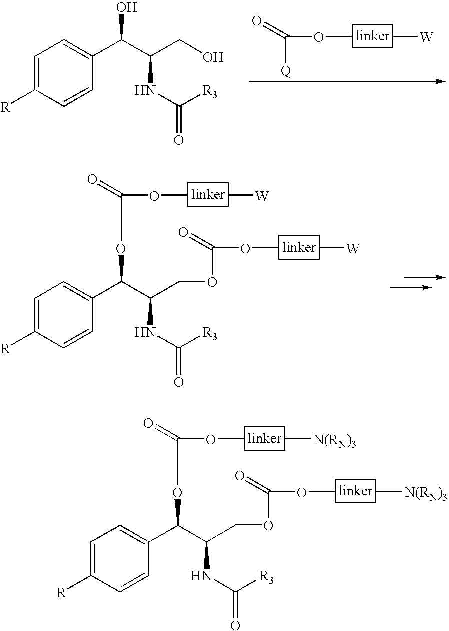

The necessary acids or chloroformates, if not commercially available, can readily be prepared by methods known to those in the art. Appropriate reaction conditions, solvents, etc., are exemplified below.

As shown in the above reaction schemes both alcohol functionalities of chloramphenicol or thiamphenicol or an analog of either are reacted with the activated carboxylic acid or alkoxy carbonic acid reagents having the nucleophilic displacement of group Q. Most common reagents of this type utilize Q=chlorine but many other leaving groups known in the art may be also employed.

As alternative to chloroformates (Q=chlorine), other reagents with different leaving groups Q may be used for preparation of carbonates. Representative references are cited for each leaving group, each incorporated by reference herein.

The reaction may be facilitated by the addition of a catalyst like a trialkylamine, pyridine, a 4-alkylpyridine, a 4-diaminoalkyl pyridine or a combination thereof. Formation of the initial ester or carbonate intermediate can be conveniently performed in a variety of solvents. Suitable solvents include, for example, chlorinated solvents such as dichloromethane and 1,2-dichloroethane; ester solvents such as ethyl acetate, isopropyl acetate, isoamyl acetate, ethylene glycol diacetate, propylene glycol diacetate, glycerol triacetate; monoether solvents such as diethyl ether, diisopropyl ether, methyl tert-butyl ether; polyether solvents such as ethylene glycol ethers, dimethyl ethylene glycol ether, diethylene glycol ethers; diethylene glycol dimethyl ether, diethylene glycol diethyl ether; formaldehyde acetal ethers such as dimethoxymethane, diethoxymethane, dibutoxymethane; cyclic ethers such as tetrahydrofuran, 1,3-dioxolane, 1,4-dioxane; ketone solvents such as acetone, methyl ethyl ketone, methyl isobutyl ketone; mixed ether/ester solvents as represented by monoethers of ethylene and diethylene glycol such as 2-methoxyethyl acetate, 2-ethoxyethyl acetate, 2-(methoxy-ethoxy)ethyl acetate, and 2-(ethoxy-ethoxy)ethyl acetate.

As used herein, “amino acid” refers to the known natural alpha-amino acids, especially those selected from alanine, cysteine, phenylalanine, glycine, histidine, isoleucine, lysine, leucine, methionine, asparagine, proline, glutamine, arginine, serine, threonine, valine, tryptophan, and tyrosine. “Amino acid side chain” and “amino acid residue” refer to a group derived from an alpha-amino acid and represents the Rₐₐ group in the NH2-CH(Rₐₐ)CO₂H structure of the amino acid, for example —CH(CH₃)₂ for valine, —CH₂CH₂CH₂CH₂NH₂ for lysine and —CH₂OH for serine. For proline it represents —H₂CH₂CH₂—which has its distal end attached to the alpha nitrogen atom. The term “alpha-N-unfunctionalized” refers to an amino acid residue with an unsubstituted —NH₂ group in the alpha position, as opposed to functionalized residues, in which for instance the alpha-amino group is a part of an amide bond of a peptide.

“Alkyl” refers to a straight or branched chain saturated-hydrocarbon moiety having the number of carbon atoms designated (i.e. C₁-C₁₀ means one to ten carbons). Examples of alkyl groups include methyl, ethyl, n-propyl, isopropyl, n-butyl, t-butyl, isobutyl, sec-butyl, and the various pentyl, hexyl, heptyl, octyl, etc. groups. Alkyl groups also include those having one or more heteroatoms in the chain, e.g., methoxymethyl (CH₃OCH₂—), ethoxyethyl, methylthiomethyl (CH₃SCH₂—), methylaminomethyl (CH₃NHCH₂—) and the like.

“Alkylene chain” refers to a divalent hydrocarbyl group derived from an alkyl group, i.e., a saturated straight or branched chain hydrocarbyl group linked at both of its ends to the remainder of the molecule in question. Typical alkylene groups include methylene, —CH₂—, ethylene, —CH₂CH₂— and n-propylene, —CH₂CH₂CH₂—. As with the alkyl groups, alkylene chains can include one or more hetero atoms, e.g., —CH₂CH₂—NH—CH₂CH₂— When an alkylene chain is combined with a nitrogen atom, as in groups having the formula NR_{y}R_{z} the overall group is a heterocyclic group such as a piperidinyl, etc group. When the alkylene chain also includes a heteroatom, the resulting group NR_{y}R_{z} would be, for instance, a cyclic moiety containing two nitrogen atoms such as a piperidinyl group.

The vehicle systems that are suitable in the compositions or formulation of this invention can be characterized as lipophilic compounds, and include a heterogeneous collection of non-polar hydrophobic materials such as vegetable animal oils (sesame, soybean, lanolin, etc), fatty acids (oleic lauric, etc), fatty acid esters (ethyl oleate, sorbitan laurate), organic acid esters (triethylcitrate, ethyltartrate, etc), alcohols (benzyl, butanol, stearyl alcohol etc), glyceryl ethers, phospholipids, terpenes and waxes.

As used herein: “A” or “An” means one, or more than one of the item in question. Thus, for instance, mention that a composition or formulation of this invention comprises “a prodrug” means that the composition may contain a single prodrug of a phenicol or may contain two or more phenicol prodrugs, including different prodrugs of the same phenicol and/or prodrugs of different phenicols. Similarly a statement that a composition or formulation of the invention comprises “a lipophilic vehicle” means that the formulation may comprise only a single lipophilic ingredient, or may comprise a combination of two or more suitable lipophilic excipient.

“About” generally signifies that a value is within twenty percent of the indicated value, unless otherwise indicated.

“Prodrug” denotes a compound that is a drug precursor which, upon administration to a subject, undergoes chemical conversion by metabolic or chemical processes to yield the active drug, e.g., a carbonate of a phenicol antibiotic is a prodrug that releases a phenicol antibiotic in vivo.

“Pharmaceutical composition” refers to a composition or formulation comprising a compound according to this invention, including pharmaceutically salts thereof, (e.g., a florfenicol prodrug) with a pharmaceutically acceptable excipient and/or carrier.

“Excipient” refers to an inert substance added to a pharmacological composition to further facilitate formulation or administration of an active ingredient. Examples, without limitation, of excipients include calcium carbonate, calcium phosphate, various sugars and types of starch, cellulose derivatives, gelatin, vegetable oils and polyethylene glycols.

“Administer” or “administration” refers to the delivery of a pharmaceutical composition or formulation of this invention to an organism for the purpose of treating or preventing a microbial infection.

“Therapeutically-effective amount,” as used herein, refers to that amount of a prodrug in a composition or formulation of the present invention that will hydrolyze sufficiently rapidly and in sufficient amounts t provide the parent phenicol in a concentration at which it can relieve to some extent one or more of the symptoms of a bacteria infection in a subject. In particular embodiment, a therapeutically-effective amount refers to that amount of a prodrug in a composition or formulation of the present invention that, when administered to a subject, delivers the parent phenicol to a subject in a sufficient plasma concentration to: (1) reduce, and preferably eliminate, the population of bacterial cells in a subject's body; (2) inhibit (i.e., slow, or preferably stop) proliferation of the bacterial cells; (3) inhibit (i.e., slow, preferably stop) spread of the bacterial infection; and/or (4) relieve (preferably eliminate) one or more symptoms associated with the infection.

“Phenicol” refers to chloramphenicol, thiamphenicol, florfenicol, and/or analogs of these compounds having other substituents on the phenyl ring, as described in Formula (I) above.

“Prophylactically-effective amount” refers to the amount of a prodrug of a phenicol of the present invention, that provides, upon hydrolysis, a sufficient plasma concentration of the phenicol to: (1) maintain a reduced level of a population of bacterial cells achieved by a previously-administered therapeutically-effective amount of the prodrug or some other appropriate drug; (2) maintain the level of inhibition of the proliferation of bacterial cells achieved by administration of a therapeutically-effective amount of a drug; (3) maintain the degree of inhibition of the spread of the infection achieved by a therapeutically-effective amount of a drug; and/or (4) maintain the level of relief of one or more symptoms, or if symptoms were eliminated, maintain the non-existence of symptoms associated with a bacterial infection achieved by administration of a therapeutically-effective amount of a prodrug (e.g., of florfenicol) of the present invention or some other appropriate drug. A prophylactically-effective amount also refers to that amount of a composition comprising a phenicol prodrug according to the present invention that will deliver the phenicol, in a sufficient plasma concentration to prohibit bacteria from accumulating in a susceptible organism in sufficient quantity to cause an infection.

“Subject” refers to an animal species capable of being infected by a pathogenic bacterium. In a particular embodiment, the animal is a human. Appropriate animal subjects also include those in the wild, livestock (e.g., raised for meat, fish, milk, butter, eggs, fur, leather, feathers and/or wool), beasts of burden, research animals, aquarium fish and companion animals (e.g., cats, dogs and rabbits) as well as those raised for/in zoos wild habitats and/or circuses. In a particular embodiment a “subject” of the invention is a “food producing” animal. For purposes of the present invention, the term “food-producing” animal shall be understood to include all animals bred for consumption, or for consumables (e.g., dairy cows, egg-laying hens and the like) by humans and/or other animals. A non-limiting list of such animals include avians (chickens, turkeys, geese, ducks, ostriches, etc.), bovines (e.g., cattle, dairy cows, buffalo), ovines (e.g., goats or sheep), porcines (e.g., hogs or pigs), equines (e.g., horses) etc., as well as aquatic animals including shellfish and fish such as trout or salmon, and other species raised or harvested for human consumption. For purposes of the present invention, the term “fish” shall be understood to include without limitation, the Teleosti grouping of fish. i.e., teleosts. Both the Salmoniformes order (which includes the Salmonidae family) and the Perciformes order (which includes the Centrarchidae family) are contained within the Teleosti grouping. Examples of potential fish recipients include the Salmonidae family, the Serranidae family, the Sparidae family, the Cichlidae family, the Centrarchidae family, the three-Line Grunt (Parapristipoma trilineatum), and the Blue-Eyed Plecostomus (Plecostomus spp).

In another embodiment, the subject is a companion animal. For purposes of the present invention, the term “companion” animal shall be understood to include housecats (feline), dogs (canine), rabbit species, horses (equine), rodents (e.g., guinea pigs, squirrels, rats, mice, gerbils, and hamsters), primates (e.g., monkeys) and avians, such as pigeons, doves, parrots, parakeets, macaws, canaries, and the like.

Other animals are also contemplated to benefit from the compositions or formulations of the present invention, including marsupials (such as kangaroos), reptiles (such as fanned turtles), game birds, swans, ratites and other economically important domestic animals.

According to the present invention, by suspending water-soluble phenicol prodrugs in a lipophilic vehicle system, it is possible to increase the duration of circulating levels of the phenicol. The initial peak concentration of the phenicol would become blunted but the circulating levels above the minimum inhibitory concentration (MIC) would last longer. This is a desirable type of pharmacokinetic profile for a phenicol type of antibiotic, particularly for those phenicols, such as florfenicol, that are termed “time-dependent” antibiotics, i.e. those types of antibiotics in which longer duration above the MIC levels is more important for efficacy than the initial elevated plasma concentrations.

Compositions of this invention typically will contain from about 10 to about 60% of one or more phenicol prodrugs, preferably from about 20 to about 40% of the phenicol prodrug or prodrugs (combined total). In particular pharmaceutical compositions containing one or more non-prodrug forms of a phenicol are also included.

The lipophilic vehicle component of the compositions generally comprises from about 40 to about 90%, preferably from about 60 to about 80%, of the composition or formulation.

The compositions or formulations of this invention will, in addition, contain excipients that are advantageous for preparing and maintaining suspensions and for injectable formulations in general. These include suspending agents such as surface-active agents/surfactants (e.g. sorbitan mono-oleate), polyvinylpyrrolidone, or poloxamers, which will be included in amounts as needed to perform their usual functions, as known in the art.

The suspensions of this invention are prepared in typical fashion for such compositions, as known in the art, by carefully mixing together the prodrug(s), lipophilic vehicle systems, suspending agents, and other ingredients. Although the order of mixing is not important, a preferred method would include adding the solid to the vehicle. The suspension can be prepared at any convenient temperature, such as ambient temperature or room temperature, however, warming the vehicle could facilitate the drug dispersion.

A therapeutically effective amount refers to an amount of compound effective to prevent and/or minimize microbial infection, and/or treat, alleviate and/or ameliorate symptoms due to a microbial infection. Determination of a therapeutically effective amount is well within the capability of those skilled in the art, especially in light of the disclosure herein.

For any compound used in the compositions and methods of the invention, the therapeutically effective amount can be estimated initially from known properties of the antibiotic agent that is released by the inventive prodrug compounds. Then, the dosage can be formulated for use in animal models so as to achieve a circulating concentration range that is at or greater than the minimum inhibitory concentration (“MIC”) as previously known to the art. Such information can then be used to more accurately determine dosages useful in patients.

Therapeutic efficacy of the compounds described herein can be determined by standard pharmaceutical procedures in cell cultures or experimental animals. For example, the minimum inhibitory concentration (“MIC”) is determined according to the guidelines laid down by the Clinical and Laboratory Standards Institute (CLSI). Similarly, the toxicity of the compounds described herein can be depicted as LD₅₀ of the compound, which is a lethal dose for 50% of subjects in a group treated with a particular compound.

As used herein the term “Minimum Inhibitor Concentration” is used interchangeably with “MIC”. An “MIC₅₀” is the concentration of the compound (e.g., the prodrug of the present invention) at which the growth of 50% of the isolates is inhibited. Similarly, MIC₉₀ is the concentration of the compound at which the growth of 90% of the isolates is inhibited.

“Metaphylaxis” is the timely mass medication of an entire group of animals to eliminate or minimize an expected outbreak of disease, e.g. in one or more animals at high risk of infection. In one particular embodiment, high risk calves are light weight, commingled with long haul cattle with unknown health histories.

The data obtained can be used to formulate a range of dosages useful in patients. The dosage, of course, may vary depending upon the dosage form and route of administration. The exact formulation, route of administration and dosage can be selected by the individual clinician in view of the subject's condition. (See e.g., Fingl, et al., 1975, in “The Pharmacological Basis of Therapeutics”, Ch. 1 p. 1). Broadly, the inventive compounds are administered to an animal in need of such treatment in a dose effective to reach and/or maintain concentrations of released antibiotic in plasma and body tissues at levels effective for the purpose, whether to treat and eliminate susceptible infectious microorganisms or to prevent new infection, for a sufficient time period to accomplish the desired goal. Those skilled in the art will appreciate that the following estimated dose ranges are adjustable based on clinical response, as well as accounting for the relative amount of the phenicol antibiotic release from each respective prodrug compound. For example, for subcutaneous administration, the inventive compounds are generally administered at a dose ranging from about 1 mg/kg to about 150 mg/kg of body weight. Frequency of administration can also range from a single dose per day to multiple doses per day. For oral administration then dose will preferably be administered once per day.

Dosage amount and interval may be adjusted individually to provide plasma levels of the compound that are sufficient to maintain a concentration above or equal to the MIC or any other desired level. Such plasma levels are often referred to as minimum effective concentrations (MECs). The MEC will vary for each compound but can be estimated from in vitro data, e.g. the concentration necessary to achieve greater than 80% inhibition of a microbial population. The MEC may be ascertained using the assays described herein. Dosages necessary to achieve the MEC will depend on the individual characteristics of the compound and/or on the animal and/or route of administration. HPLC assays or bioassays can be used to determine plasma concentrations of the compound and/or its corresponding active product.

Dosage intervals can also be determined using MEC value. Compounds should be administered using a regimen that maintains plasma levels above the MEG for 10-90% of the time.

In cases of local administration or selective uptake, the effective local concentration of the drug may not be related to plasma concentration. Other procedures known in the art may be employed to determine the correct dosage amount and interval.

The compositions may be administered once daily or divided into multiple doses. Often only one dose will be sufficient to treat the infection. In some circumstances one dose followed by a second dose 48 hours later will be required to treat the animal. The precise dose will depend on the stage and severity of the infection, the susceptibility of the infecting organism to the composition, and the individual characteristics of the animal species being treated, as will be appreciated by one of skill in the art.

The amount of a composition administered will, of course, be dependent on the patient being treated, pathogen or bacteria causing the infection, the severity of the infection, the manner of administration, i.e., oral, intravenous, topical, etc., and the judgment of the prescribing physician, veterinarian, etc.

The inventive compositions will generally be administered so as to provide the prodrug at a dose ranging from about 1 mg to about 150 mg/kg body weight in cattle, when using the subcutaneous route. Preferably, the dose ranges from about 20 mg to about 70 mg/kg body weight. More preferably the dose ranges from about 45 to about 65 mg/kg; most preferably it is about 60 mg/kg. However, when the inventive compound is administered via the intra-muscular (IM) route, the dose is preferably administered twice, with the administration of the second dose being about 24 to about 48 hours after the administration of the first dose.

An embodiment of the invention includes methods of eliminating, reducing or preventing bacterial infections in fish, and optionally aquatic invertebrates. The methods include administering an effective amount of a compound of the invention to an aquatic animal in need thereof. Administering may be achieved by injection the fish or other aquatic animal with the composition.

The dose of the inventive compounds that is effective for reducing, eliminating, or preventing the bacterial infection in fish or other aquatic species can be routinely determined by a veterinarian using the parameters and methods discussed supra for other types of animals, although it may vary depending on the species of fish treated, the particular microorganisms involved, and the degree of infection. For aquaculture indications, the inventive compounds will generally be administered at a dosage of about 1 mg/kg to about 70 mg/kg, and preferably from 10 mg/kg to 30 mg/kg. Suitable routes of administering include: intravenously, subcutaneously, intramuscularly and/or by spraying or dipping the aquatic species as needed, and/or by directly adding the compound into the water in a holding volume.

It is also contemplated to administer the inventive prodrug compounds in combination, simultaneously, or sequentially (e.g. in the same composition or in separate compositions) with other useful art-known medicinal agents. Such medicinal agents include, e.g., other microbiocides, e.g., antibiotics, antifungals, antivirals, parasiticides, and so forth, as well as in nutritional supplements, feed additives and the like. For example, it is contemplated to administer any art-known standard (non-prodrug) phenicol such as florfenicol, chloramphenicol or thiamphenicol themselves in combination with the inventive compounds. Processes for the manufacture of these antibiotic compounds, and intermediates useful in such processes, are described in U.S. Pat. Nos. 4,311,857; 4,582,918; 4,973,750; 4,876,352; 5,227,494; 4,743,700; 5,567,844; 5,105,009; 5,382,673; 5,352,832; ad 5,663,361, all of which are hereby incorporated by reference. Other florfenicol analogs and/or prodrugs have been disclosed and such analogs also can be used in the compositions and methods of the present invention [see e.g., U.S. Patent Application Publication No. 2004/0082553, and U.S. Pat. No. 7,153,842, both of which are hereby incorporated by reference in their entireties]. When the antibiotic compound is florfenicol, the concentration of florfenicol typically is from about 10% to about 50%, with the preferred level between about 20% and about 40%, even more preferred being at least about 30% (in these cases, given as w/w in the case of solid compositions and w/v in the case of liquid compositions).

Another useful antibiotic compound for use in a combination with the inventive compounds is tilmicosin. Tilmicosin is a macrolide antibiotic that is chemically defined as 20-dihydro-20-deoxy-20-(cis-3,5-dimethylpiperidin-1-yl)-desmycosin and which is reportedly disclosed in U.S. Pat. No. 4,820,695, hereby incorporated by reference. Also disclosed in U.S. Pat. No. 4,820,695 is an injectable, aqueous formulation comprising 50% (by volume) propylene glycol, 4% (by volume) benzyl alcohol, and 50 to 500 mg/ml of active ingredient. Tilmicosin may be present as the base or as a phosphate. Tilmicosin has been found to be useful in treatment of respiratory infections, particularly Pasteurella haemolytica infections in cattle when administered by injection over a 4 day treatment period. Accordingly, tilmicosin may be used in treatment of, for example, neonatal calf pneumonia and bovine respiratory disease. When tilmicosin is present, it is present in an amount of about 1% to about 50% w/v, preferably 10% to about 50%, and in a particular embodiment, 30%.

Another useful antibiotic for use in combination with the inventive compounds is tulathromycin. Tulathromycin may be prepared in accordance with the procedures set forth in U.S. Patent Publication No. 2003/0064939 A1, which is hereby incorporated by reference in its entirety. Tulathromycin may be present in injectable dosage forms at concentration levels ranging from about 5.0% to about 70% by weight. Tulathromycin is most desirably administered in dosages ranging from about 0.2 mg per kg body weight per day (mg/kg/day) to about 200 mg/kg/day in single or divided doses (i.e., from 1 to 4 doses per day), and more preferably 1.25, 2.5 or 5 mg/kg once or twice weekly, although variations will necessarily occur depending upon the species, weight and condition of the subject being treated. Tulathromycin may be present in injectable dosage forms at concentration levels ranging from about 5.0% to about 70% by weight (w/v).

Another useful antibiotic for use in combination with the inventive compounds is the fluoroquinolones family of antibiotics, such as, for example, enrofloxacin, danofloxacin, difloxacin, orbifloxacin and marbofloxacin. In the case of enrofloxacin, it may be administered in a concentration of about 100 mg/ml, danofloxacin may be present in a concentration of about 180 mg/ml.

Other useful macrolide antibiotics for use in combination with the inventive compounds include compounds from the class of ketolides, or, more specifically, the azalides. Such compounds are described in, for example, U.S. Pat. Nos. 6,514,945, 6,472,371, 6,270,768, 6,437,151 and 6,271,255, U.S. Pat. Nos. 6,239,112, 5,958,888, 6,339,063 and 6,054,434, all of which are hereby incorporated by reference in their entireties.

Other useful antibiotics for use in combination with the inventive compounds include the tetracyclines, particularly chlortetracycline and oxytetracycline.

Other antibiotics may include beta-lactams such as one of the penicillins, e.g., penicillin G, penicillin K, ampicillin, amoxicillin, or a combination of amoxicillin with clavulanic acid or other beta-lactamase inhibitors. Additional particular beta-lactams include the cephalosporins such as, for example, ceftiofur, cefquinome, etc.

For all of the methods and the inventive compounds described herein, it is also contemplated that the identified compounds are readily employed in combination with one or more art-known agents for killing or controlling various types of parasites, e.g., including all of the ecto- and endoparasites described herein. Thus, although the inventive compounds and methods are preferred over previously known agents and methods of using previously known agents, in certain optional embodiments they are contemplated to be employed in combination, simultaneously, or sequentially (e.g. in the same composition or in separate compositions), with other art-known agents or combinations of such art-known agents employed for killing or controlling various types of pests.

These additional agents for use in combination with the inventive compounds include, for example, art-known anthelmintics, such as, for example, avermectins (e.g. ivermectin, moxidectin, milbemycin), benzimidazoles (e.g. albendazole, triclabendazole), salicylanilides (e.g. closantel, oxyclozanide), substituted phenols (e.g. nitroxynil), pyrimidines (e.g. pyrantel), imidazothiazoles (e.g. levamisole) and praziquantel.

Additional art-known agents for killing or controlling pests for use in combination with the inventive compounds include the organophosphate pesticides. This class of pesticides has very broad activity, e.g. as insecticides and, in certain instances, anthelmintic activity. Organophosphate pesticides include, e.g., dicrotophos, terbufos, dimethoate, diazinon, disulfoton, trichlorfon, azinphos-methyl, chlorpyrifos, malathion, oxydemeton-methyl, methamidophos, acephate, ethyl parathion, methyl parathion, mevinphos, phorate, carbofenthion, phosalone, to name but a few such compounds. It is also contemplated to include combinations of the inventive methods and compounds with carbamate type pesticides, including, e.g., carbaryl, carbofuran, aldicarb, molinate, methomyl, etc., as well as combinations with the organochlorine type pesticides. It is further contemplated to include combinations with biological pesticides, including e.g. repellents, the pyrethrins (as well as synthetic variations thereof, e.g., allethrin, resmethrin, permethrin, tralomethrin), and nicotine, that is often employed as an acaricide. Other contemplated combinations are with miscellaneous pesticides including: Bacillus thuringiensis, chlorobenzilate, formamidines, (e.g. amtitaz), copper compounds, e.g., copper hydroxide, cupric oxychloride sulfate, cyclotron, cypermethrin, dicofol, endosulfan, esenfenvalerate, fenvalerate, lambda-cyhalothrin, methoxychlor and sulfur.

In addition, for all of the methods and new compounds described herein, it is further contemplated that the identified compounds can be readily employed in combination with synergists such as piperonyl butoxide (PBO) and triphenyl phosphate (TPP); and/or with Insect Growth Regulators (IGRs) and Juvenile Hormone Analogues (JHAs) such as diflubenzuron, cypromazine, methoprene, etc., thereby providing both initial and sustained control of parasites (at all stages of insect development, including eggs) on the animal subject, as well as within the environment of the animal subject.

Combinations with cyclodienes, ryania, KT-199 and/or older art-known anthelmintic agents, such as avermectins (e.g., ivermectin, moxidectin, milbemycin), benzimidazoles (e.g., albendazole, triclabendazole), salicylanilides (e.g., closantel, oxyclozanide), substituted phenols (e.g., nitroxynil), pyrimidines (e.g., pyrantel), imidazothiazoles (e.g., levamisole), praziquantel and some organophosphates such as naphthalophos and pyraclofos, are also contemplated to be employed in such combinations.

In particular, additional antiparasitic compounds useful within the scope of the present invention are preferably comprised of the class of avermectin compounds. As stated above, the avermectin family of compounds is a series of very potent antiparasitic agents known to be useful against a broad spectrum of endoparasites and ectoparasites in mammals.

A preferred compound for use in combination with the inventive compounds within the scope of the present invention is ivermectin. Ivermectin is a semi-synthetic derivative of avermectin and is generally produced as a mixture of at least 80% 22,23-dihydroavermectin B1ₐ and less than 20% 22,23-dihydroavermectin B1_{b}. Ivermectin is disclosed in U.S. Pat. No. 4,199,569, hereby incorporated by reference. Ivermectin has been used as an antiparasitic agent to treat various animal parasites and parasitic diseases since the mid-1980s.

Abamectin is an avermectin that is disclosed as avermectin B1a/B1 b in U.S. Pat. No. 4,310,519, which is hereby incorporated by reference in its entirety. Abamectin contains at least 80% of avermectin B1ₐ and not more than 20% of avermectin B1_{b}.

Another preferred avermectin is doramectin also known as 25-cyclohexyl-avermectin B₁. The structure and preparation of doramectin, is disclosed in U.S. Pat. No. 5,089,480, which is hereby incorporated by reference in its entirety.

Another preferred avermectin is moxidectin. Moxidectin, also known as LL-F28249 alpha is known from U.S. Pat. No. 4,916,154, which is hereby incorporated by reference in its entirety.

Another preferred avermectin is selamectin. Selamectin is 25-cyclohexyl-25-de(1-methylpropyl)-5-deoxy-22,23-dihydro-5-hydroxyimino)-avermectin B₁ monosaccharide.

Milbemycin, or B41, is a substance which is isolated from the fermentation broth of a milbemycin producing strain of Streptomyces. The microorganism, the fermentation conditions and the isolation procedures are more fully described in U.S. Pat. Nos. 3,950,360 and 3,984,564.

Emamectin (4″-deoxy-4″-epi-methylaminoavermectin B₁), which can be prepared as described in U.S. Pat. No. 5,288,710 or 5,399,717, is a mixture of two homologues, 4″-deoxy-4″-epi-methylaminoavermectin B1a and 4″-deoxy-4″-epi-methylaminoavermectin B1b. Preferably, a salt of emamectin is used. Non-limiting examples of salts of emamectin which may be used in the present invention include the salts described in U.S. Pat. No. 5,288,710, e.g., salts derived from benzoic acid, substituted benzoic acid, benzenesulfonic acid, citric acid, phosphoric acid, tartaric acid, maleic acid, and the like. Most preferably, the Emamectin salt used in the present invention is emamectin benzoate.

Eprinomectin is chemically known as 4″-epi-Acetylamino-4″-deoxy-avermectin B₁. Eprinomectin was specifically developed to be used in all cattle classes and age groups. It was the first avermectin to show broad-spectrum activity against both endo- and ecto-parasites while also leaving minimal residues in meat and milk. It has the additional advantage of being highly potent when delivered topically.

The compositions of the present invention optionally comprise combinations of one or more of the following antiparasite compounds (parasiticides) (all patents and published patent applications mentioned below are hereby incorporated herein in their entireties):
The antiparasite imidazo[1,2-b]pyridazine compounds as described by U.S. Patent Application Publication No. 2005/0182059.
The antiparasite 1-(4-mono and di-halomethylsulphonylphenyl)-2-acylamino-3-fluoropropanol compounds, as described by U.S. Patent Application Publication No. 2005/0182139.
The antiparasite trifluoromethanesulfonanilide oxime ether derivative compounds, as described by U.S. Patent Application Publication No. 2006/0063841.
The antiparasite phenyl-3-(1H-pyrrol-2-yl)acrylonitrile compounds, as described by U.S. Patent Application Publication No. 2006/0128779.
The antiparasite N-[(phenyloxy)phenyl]-1,1,1-trifluoromethanesulfonamide and N-[phenylsulfanyl)phenyl]-1,1,1-trifluoromethanesulfonamide derivatives, as described by U.S. Patent Application Publication No. 2006/0281695.
The antiparasite N-phenyl-1,1,1-trifluoromethanesulfonamide hydrazone compounds, as described by U.S. Patent Application Publication No. 2007/0238700.

The compositions of the present invention may also be employed in combination with a flukicide. Suitable flukicides include, for example, triclabendazole, fenbendazole albendazole, clorsulon and oxibenidazole. It will be appreciated that the above combinations may further include combinations of antibiotic, antiparasitic and anti-fluke active compounds.

In addition to the above combinations, it is also comtemplated to provide combinations of the inventive methods and compounds, as described herein, with other animal health remedies such as trace elements, anti-inflammatories, anti-infectives, hormones, dermatological preparations, including antiseptics and disinfectants, and immunobiologicals such as vaccines and antisera for the prevention of disease.

For example, such antiinfectives include one or more antibiotics that are optionally co-administered during treatment using the inventive compounds or methods, e.g., in a combined composition and/or in separate dosage forms. Art-known antibiotics suitable for this purpose include, for example, those listed hereinabove.

Further, it is also contemplated that the inventive methods and compounds be advantageously employed in combination, simultaneously or sequentially, with art-known animal health remedies e.g., trace elements, vitamins, anti-inflammatories, anti-infectives and the like, in the same or different compositions.

Suitable anti-inflammatory agents include, e.g., both steroidal and non-steroidal anti-inflammatory agents.

Non-steroidal anti-inflammatory agents, including their racemic mixtures or individual enantiomers where applicable, can include ibuprofen, flurbiprofen, ketoprofen, aclofenac, diclofenac, aloxiprin, aproxen, aspirin, diflunisal, fenoprofen, indomethacin, mefenamic acid, naproxen, phenylbutazone, piroxicam, salicylamide, salicylic acid, sulindac, desoxysulindac, tenoxicam, tramadol, ketoralac, flufenisal, salsalate, triethanolamine salicylate, aminopyrine, antipyrine, oxyphenbutazone, apazone, cintazone, flufenamic acid, clonixeril, clonixin, meclofenamic acid, flunixin, colchicine, demecolcine, allopurinol, oxypurinol, benzydamine hydrochloride, dimefadane, indoxole, intrazole, mimbane hydrochloride, paranylene hydrochloride, tetrydamine, benzindopyrine hydrochloride, fluprofen, ibufenac, naproxol, fenbufen, cinchophen, diflumidone sodium, fenamole, flutiazin, metazamide, letimide hydrochloride, nexeridine hydrochloride, octazamide, molinazole, neocinchophen, nimazole, proxazole citrate, tesicam, tesimide, tolmetin, and triflumidate.

In a particular embodiment, a compound of the present invention is employed in combination with flunixin, [see, e.g., U.S. Pat. No. 6,790,867 B2, which is hereby incorporated by reference in its entirety.] In a related embodiment, the present invention provides a pharmaceutical composition comprising a compound of the present invention and flunixin.

Steroidal anti-inflammatory agents include, for example, glucocorticoid agents such as dexamethasone, cortisone, hydrocortisone, prednisone, beclomethasone, betamethasone, flunisolide, methyl prednisone, para methasone, prednisolone, triamcinolome, alclometasone, amcinonide, clobetasol, fludrocortisone, difluorosone diacetate, fluocinolone acetonide, fluoromethalone, flurandrenolide, halcinonide, medrysone, mometasone, and pharmaceutically acceptable salts and mixtures thereof.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A pharmaceutical composition comprising a therapeutically effective amount of a water-soluble prodrug of a phenicol, wherein the composition comprises a suspension of the phenicol prodrug in a lipophilic vehicle system.

2. The pharmaceutical composition of claim 1 wherein the water-soluble phenicol prodrug is a florfenicol prodrug.

3. The pharmaceutical composition of claim 1 wherein the prodrug has the formula (II): 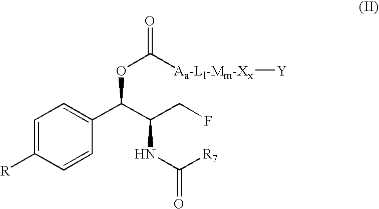
in which:
R is selected from the group consisting of 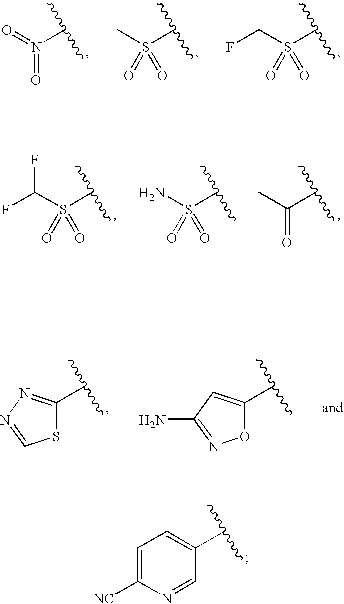
A is oxygen and a is zero or 1;
L is (a) CH₂ and l is an integer from 1 to 6; (b) CHR₁ where R₁ is an amino acid side chain and l is 1; or (c) CHR₁NHC(O)CH(NH₂)R₂ where R₁ and R₂ are amino acid side chains and l is 1;
M is (a) oxygen or sulfur and m is zero or one; (b) CH₂ and m is zero or an integer from 1 to 4; or (c) and m is 1;
X is (a) CH₂ and x is zero or an integer from 1 to 4; or (b) C(O) and x is 1; and Y is (a) NH₂; (b) NHRₓ where Rₓ is methyl, ethyl, n-propyl or isopropyl; (c) NR_{y}R_{z}, where R_{y} and R_{z} are independently hydrogen, methyl, ethyl, n-propyl or isopropyl, or R_{y} and R_{z} taken together from a C₂-C₅ alkylene chain, or a C₂-C₄ alkylene chain further including a nitrogen or oxygen heteroatom in said chain; (d) C(═NH)NH₂; (e) N⁺R₄R₅R₆ where R₄, R₅ and R₆ are independently hydrogen, methyl or ethyl, or R₄ and R₅ taken together form a C₂-C₅, alkylene chain, or a C₂-C₄ alkylene chain further including a nitrogen or oxygen heteroatom in said chain; (f) pyridinium; (g) N-methyl or N-ethyl pyridinium; (h) N′-3-methyl-N-1-imidazolium; (i) a phenyl group substituted by a group having the formula NR₄R₅ or N⁺R₄R₅R₆ where R₄, R₅ and R₆ are as defined above; or (j) NH—CR₃(═NH) where R₃ is hydrogen, methyl or amino;
and R₇ is selected from the group consisting of dichloromethyl, difluoromethyl, trifluoromethyl, cyanomethyl, azidomethyl, and aminomethyl;
provided that the group AₐLₗMₘXₓY is other than an alpha-N-unfunctionalized glycine, ornithine or lysine residue;
and pharmaceutically acceptable salts thereof.

4. The pharmaceutical composition of claim 1 wherein the prodrug has the formula (III): 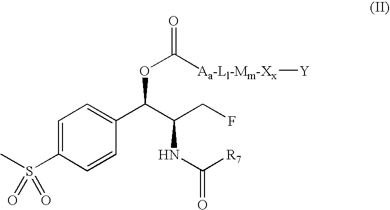
in which:
A is oxygen and a is zero or 1:
L is (a) CH₂ and l is an integer from 1 to 5 or (b) CHR₁ where R₁ is an amino acid side chain and l is 1; or (c) CHR₁NHC(O)CH(NH₂)R₂ where R₂ is an amino acid side chain and l is 1;
M is (a) oxygen and m is zero or one, (b) CH₂ and m is zero or an integer from 1 to 4; or (c) NH and m is 1;
X is (a) CH₂ and x is zero or an integer from 1 to 4; or (b) C(O) and x is 1; and
Y is (a) NH₂; (b) NHRₓ where Rₓ is methyl, ethyl, n-propyl or isopropyl; (c) NR_{y}R_{z} where R_{y} and R_{z} are independently hydrogen, methyl, ethyl, n-propyl or isopropyl (d) C(═NH)NH₂; (e) N⁺R₄R₅R₆ where R₄, R₅ and R₆ are independently hydrogen, methyl or ethyl; (N-pyridinium; (g) N′-3-methyl-N-1-imidazolium; or (h) NH—CR₃(═NH) where R₃ is hydrogen, methyl or amino;
and R₇ is selected from the group consisting of dichloromethyl, difluoromethyl, trifluoromethyl, cyanomethyl, azidomethyl, and aminomethyl; (with R₇ preferably being dichloromethyl)
provided that the sum of a+l+m+x is from 2 to 6 and preferably from 3 to 6;
provided that if a is 1, then M is (CH₂)ₘ;
and provided that the group AₐLₗMₘXₓY is other than an alpha-N-unfunctionalized glycine, ornithine or lysine residue;
and pharmaceutically acceptable salts thereof.

5. The pharmaceutical composition of claim 1 wherein the prodrug has the formula (IV): 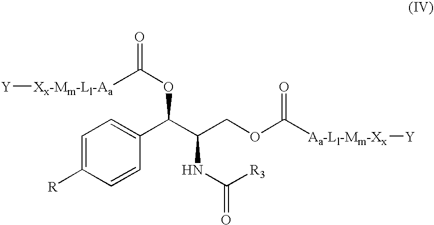
in which:
R is selected from the group consisting of 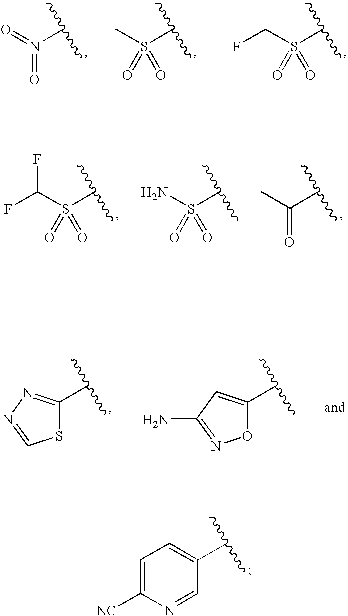
A is oxygen and a is zero or 1;
L is (a) CH₂ and l is an integer from 1 to 6; (b) CHR₁ where R₁ is an amino acid side chain and l is 1; or (c) CHR₁NHC(O)CH(NH₂)R₂ where R₁ and R₂ are amino acid side chains and l is 1;
M is (a) oxygen or sulfur and m is zero or one; (b) CH₂ and m is zero or an integer from 1 to 4; or (c) NH and nm is 1;
X is (a) CH₂ and x is zero or an integer from 1 to 4; or (b) C(O) and x is 1; and
Y is (a) NH₂: (b) NHRₓ where Rₓ is methyl, ethyl, n-propyl or isopropyl; (c) NR_{y}R_{z} where R_{y} and R_{z} are independently hydrogen, methyl, ethyl, n-propyl or isopropyl, or R_{y} and R_{z} taken together form a C₂-C₅ alkylene chain, or a C₂-C₄ alkylene chain further including a nitrogen or oxygen heteroatom in said chain; (d) C(═NH)NH₂; (e) N⁺R₄R₅R₆ where R₄, R₅ and R₆ are independently hydrogen, methyl or ethyl or R₄ and R₅ taken together form a C₂-C₅ alkylene chain, or a C₂-C₄ alkylene chain further including a nitrogen or oxygen heteroatom in said chain; (f) (pyridinium: (g) N-methyl or N-ethyl pyridinium; (h) N′-3-methyl-N-1-imidazolium; (i) a phenyl group substituted by a group having the formula NR₄R₅ or N⁺R₄R₅R₆ where R₄, R₅ and R₆ are as defined above; or (j) NH—CR₈(═NH) where R₈ is hydrogen, methyl or amino; and
R₃ is selected from the group consisting of dichloromethyl, difluoromethyl, chlorofluoromethyl, chloromethyl, methyl, cyanomethyl, azidomethyl, and aminomethyl;
and pharmaceutically acceptable salts thereof.

6. The pharmaceutical composition of claim 1 wherein the lipophilic vehicle system is selected from the group consisting of sesame oil, soybean oil, lanolin, oleic acid, lauric acid, ethyl oleate, sorbitan laurate, triethyl citrate, ethyl tartrate, benzyl alcohol, butanol, stearyl alcohol, a glyceryl ethers, a phospholipid, a terpene, a wax, and any combination thereof.

7. The pharmaceutical composition of claim 6 wherein the lipophilic vehicle system is ethyl oleate.

8. A method of treating or preventing a disease or disorder in a subject comprising administering to said subject a therapeutically effective amount of the pharmaceutical composition of claim 1.
